(19) United States
(12) Patent Application Publication
(10) Pub. No.: US 2025/0303031 A1
(43) Pub. Date: Oct. 2, 2025

(54) CLOSED AUTOMATED SYSTEM AND METHOD FOR MULTIPLEX CELL PROCESSING

(71) Applicant: Diagnologix, LLC, San Diego, CA (US)

(72) Inventors: Miguel Angel Lopez Ramirez, San Diego, CA (US); Shengdong Liu, San Diego, CA (US); Yu-Tsueng Liu, San Diego, CA (US)

(21) Appl. No.: 18/618,922

(22) Filed: Mar. 27, 2024

(51) Int. Cl. A61M 1/02

(52) U.S. Cl. CPC A61M 1/029

(57) ABSTRACT

A closed system for blood sample processing to separate biological components, and methods of cell selection from a blood sample. The system has a centrifuge having a rotor, a processing chamber containing a blood sample, and a piston housed in the processing chamber. A plurality of tubes connects the processing chamber the blood sample. Actuation of the piston dispels fractions of the blood sample during centrifugation or at rest to at least one fraction bag. The processing chamber may be moved between a horizontal orientation that is assumed during centrifugation, and a vertical position while no centrifugation is performed.

### FIELD OF THE INVENTION

This present disclosure discusses an engineered PLAX (parallel automated closed system) system, that can perform parallel multi-sample centrifugation in a fashion familiar to most biomedical researchers and staff. One aspect of PLAX involves controlling a piston inside a syringe-processing chamber using an external hydraulic pump. The present disclosure enables conventional laboratory methods to be intuitively and seamlessly executed as closed automated processes. The system may be configured to perform a variety of current laboratory routines as a closed automated process, including processes such as swinging bucket centrifugation, fixed angle centrifugation and multi-well plate centrifugation.

### BACKGROUND OF THE INVENTION

Cell processing is essential for promising cell therapies, such as tissue regeneration with stem cells and cancer treatment with cell-based immunotherapy. Cell processing may involve a variety of procedures for isolation, expansion, differentiation, genetic modification, and preservation. The cell manufacturing process requires strict adherence to standard operating procedures by highly skilled personnel working in GMP cleanrooms equipped with various complex and expensive instruments. Conventional methods for handling cells are mainly open processes, such as liquid transfer using pipettes, cells washing, and concentration using centrifuges. The risk of sample cross contamination is substantial, evident by pervasive false identities of reported cell lines in the research literature over the past several decades. A common solution to avoid this issue is to limit sample processing to only one at a time at the same location, which cannot efficiently scale out parallelly processing multiple cell production lots in a shared environment. Nevertheless, it is very challenging to perform certain crucial tasks in a closed system, which may otherwise be simple when conducted in open environments.

Density gradient-based centrifugation is a very common and useful procedure for research laboratories in open environments, but a completely different methodology is required for its application for enriching cells of interest in a closed system for clinical translation. For example, transplantation of allogeneic pancreatic islets to restore insulin-secreting β cell mass had limited success until the development of the Edmonton Protocol in 2000, which applied the Ricordi method for semi-automatic cell processing, including an enrichment step enabled by density gradient-based centrifugation using COBE 2991 cell apheresis system. This therapeutic strategy has been used to treat over 1,500 patients in about 40 centers since 2000, with 50-70% of patients achieving insulin independence at 5 years. Furthermore, Lantidra (donislecel), the first microencapsulated allogeneic islet cell therapy for type I diabetes, has recently been approved by the U.S. FDA in 2023. Perhaps the most common use of gradient-based separation is isolation of mononuclear cells from blood and bone marrow. Other applications include isolation of mesenchymal stem cells and tumor infiltrating lymphocytes.

In addition to COBE 2991 (Patent U.S. Pat. No. 3,737,096A), other automated devices have been applied for gradient-based cell separation. Sepax (Patent U.S. Pat. No. 6,733,433B1) is a popular device for this purpose. Unlike conventional density gradient-based separation (DGBS) conducted in open environments for multiple sample processing, current automated devices for DGBS in closed systems can only process one sample each time. Notably, the design and operation of the devices for achieving closed processing are very different from the more familiar centrifuges in most biological laboratories, posing potential obstacles for clinical translation of promising research protocols.

There are two main types of designs for overcoming tubing twisting while spinning for automated closed system centrifuges, namely rotating seal and “skip rope” (or seal-less). A rotating seal must have adequate friction to prevent leakage, but not too much so as to cause overheating. Moreover, the complexity and cost of rotating seal increases considerably going from two to multiple passages. Thus, several modern apheresis equipment adopts the seal-less type of anti-twisting centrifuges. It has been demonstrated that a bundle of flexible tubes forming a “skip-rope” loop remains free of twisting when connected to a rotating bowl at one end and immobilized at the other end, wherein the bowl rotates at an angular velocity of 20 around the vertical axis and the loop simultaneously revolves at @ around the same axis, as described in U.S. Patent Publication Nos. U.S. Pat. Nos. 3,586,413 A, 4,425,112 A, 4,900,298 A. This configuration been applied previously for apheresis and other devices with a single chamber.

### SUMMARY OF THE INVENTION

In one general aspect, a closed system for blood sample processing to separate biological components may include: a centrifuge having a rotor, said centrifuge being configured to centrifugate a blood sample; a processing chamber attached to the rotor, said processing chamber having a first end and a second end, and is configured to contain at least one blood sample; a piston housed in the processing chamber, said piston configured to move between the first position closer to a first end of the processing chamber and second position closer to a second end of the processing chamber, the second end being located opposite of the first end; and a plurality of tubes connecting the processing chamber to the at least one blood sample; wherein actuation of the piston making the piston move dispels fractions of the blood sample during centrifugation or at rest to at least one fraction bag.

In a second general aspect, a method of cell selection from a blood sample may include: a) priming the system by removing air from processing chamber through actuation of piston in a first direction via a hydraulic pump; b) pumping blood into the processing chamber, wherein said processing chamber is housed in a swinging bucket cassette; c) pumping density gradient separation media into the processing chamber; d) actuating centrifugal rotation of rotor to which the swinging bucket cassette is attached; and while performing centrifugation, moving a piston within the processing chamber, performing in sequence the following steps: e) extracting erythrocytes from the processing chamber; f) extracting density gradient separation media from the processing chamber; g) extracting mononuclear cells from the processing chamber; h) extracting plasma from the processing chamber; i) ceasing centrifugation, closing valves, and stabilizing pressure.

In a third general aspect, a method of cell selection from a blood sample may include: a) priming the system by removing air from processing chamber through actuation of piston in a first direction via a hydraulic pump; b) pumping blood into the processing chamber, wherein said processing chamber is housed in a swinging bucket cassette; c) pumping density gradient separation media into the processing chamber; d) actuating centrifugal rotation of rotor to which the swinging bucket cassette is attached; and while performing centrifugation, with the processing chamber in a horizontal orientation, moving a piston within the processing chamber, performing in sequence the following steps: e) extracting erythrocytes from the processing chamber; f) extracting density gradient separation media from the processing chamber; g) stopping centrifugation; h) flipping an orientation of the processing chamber with respect to the rotor so that an end of the processing chamber that was proximal to the rotor now becomes the free distal end of the processing chamber and an end of the processing chamber that was the distal free end of the processing chamber now become proximal to the rotor; i) extracting plasma from the processing chamber; j) ceasing centrifugation, closing valves, and stabilizing pressure, leaving mononuclear cells in the processing chamber.

### DETAILED DESCRIPTION OF THE INVENTION

According to a first aspect of the present disclosure, the system may be structured to allow fluids to flow in and out of a processing chamber either when the system is stationary or under centrifugation, thereby allowing fractions of different densities to be extracted or separated.

According to a second aspect of the present disclosure, the system may be configured to work like conventional swing-bucket centrifuges, but in a closed system. Therefore, as the piston moves and advances, either at spinning or rest, the order of the cell layers that are retained are expelled.

According to a third aspect of the present disclosure, the processing chamber may be accommodated at two different orientations within the swinging bucket cassette, thus facilitating expelling of only the desired fractions. This accommodation provides vast flexibility and greater possibilities for its widespread integration into other cell processing workflows, such as density gradient based separation (DGBS). This may allow for accomplishment of complete automation without human intervention, and diverse applications not previously contemplated, such as the novel microbubble-based cell separation, activation, expansion, and transduction, as described in U.S. Patent No. U.S. Pat. No. 10,479,976 B2 and U.S. Patent Application Publication No. US20220154150 A1.

The present disclosure proposes a fully closed automated system capable of cell processing and cell isolation. The system has a closed and sterile single set tubing kit that is disposable, therefore avoiding the likelihood of cross contamination. The preferred embodiment of the present disclosure includes a pre-assembled kit including a processing chamber (comprising a medical grade syringe barrel) connected to a set of tubing lines with connectors for the collection of the separated components and fractions. The chamber can be placed within the centrifuge rotor prior to spinning. The buffer bags for washing protocols are generally pre-connected to the disposable set, but if needed, they may be connected via a filter. A blood bag or other reagents and media can be connected using a sterile connecting device.

The bundle of tubing lines that route into each of the separated bag fractions is possible due to the integration of either individual or multi-channel pinch valves. In a preferred embodiment of the present disclosure, sterility of operating cells and reagents is maintained through a functionally closed system employing components that do not have any direct contact with the fluids being processed. Thus, all fluid/sample processing is strictly conducted within the confines of medical grade tubing, and its manipulation is performed using noninvasive peristaltic pumps and pinch valves. The system arrangement and architecture allow each process to use disposable tubing kits that run through retaining channels within each pinch valve, providing a tight grip for routing fluid, and eliminating any cross contamination between adjacent lines.

In a preferred embodiment, the procedure of moving fluid in and out of the processing chamber during centrifugation or stationary is made possible due to the integration of a hydraulic pump. The hydraulic system is a plunger control subsystem comprising weight cells, stepper motors, and a processing buffer syringe, along with solenoid pinch valve plates/holders with retaining channels to route the fluid flow from a premixing subsystem, or reagent bags (blood, density gradient media) through a bundle of tubes within the system to the desired container or processing chamber. As the hydraulic piston moves down, the processing chamber piston moves downwards, and vice versa.

This system, like an apheresis machine, can be categorized in three subsystems or modules that are required for cell isolation: the sorting module (Ms), control module (Mc) and electronics module (Me). The system is tailored for fully automation through the incorporation of standard and unique components for microbubble-based technologies, as described in U.S. Patent Publication No. U.S. Pat. No. 10,479,976 B2, and for cell processing. Components include a centrifuge, peristaltic pumps, pinch valves, actuators, and diverse sensors such as: ultrasonic range finders, optical sensors, imbalance sensors, weight cells, pressure sensors, and stepper motors. The control module (Mc) comprises all necessary components for fluid management, logic, and operator interfacing. The sorting module (Ms) includes the primary processing chamber for the isolation process. A primary function of the sorting module is to provide mixing of input reagents (targeted microbubbles, blood, cells, or density gradient media), and accurate control of the volume for each step of the isolation process. The electronics module (Me) contains all the physical components of the system, which are controlled with microcontrollers and chips, ensuring all the hardware components operate within their specified voltage and clock rate. The control system helps with data transmission, parallel processing, and handling interruptions. Users can operate the system via a graphical user interface (GUI) that is displayed on a built-in screen.

Processing target cells bound by microbubbles involves floating them to the liquid surface, thereby separating them from the remaining cells that sink. In a preferred embodiment, a centrifugation system may be used to expedite the process, especially for pelleting unbound cells whose densities are close to that of the buffer solution. The centrifugation system employs a seal-less anti-twisting centrifuge process that allows the system to spin the rotor base, while continuously flowing reagents in and out of a main processing chamber. A swinging bucket cassette holds a syringe-like isolated container, namely a processing chamber, that is detachable and openable for easy installation and connection of the single-use tubing set kit. When the centrifuge stops, the swinging cassette can be vertically rotated to facilitate mixing samples (e.g., microbubbles and target cells) via the use of a mixing device. For automation, the PLAX system integrates sensors to situate the centrifuge rotor to a fixed position so that the cassette and the mixing device can be correctly aligned. Additionally, the system may integrate built-in triple axis accelerometers, allowing the monitoring of the stability and imbalance of the process when subjected to any abrupt, sudden, or unexpected external factor.

A further aspect and application of this disclosure is the streamlined production of multiple CAR-T cells in parallel, manufactured by microbubble-based technologies in a complete closed environment, thereby overcoming the recurrent cross contamination issues common in open systems. This separation system and method can increase sample processing throughput, while also lowering the manufacturing labors and costs. In contrast to a single-plex machine that requires multiple machines to increase processing throughput, the dimensions and housing space do not change significantly for a multiplex machine. The simple and streamlined processing system of microbubble-based technologies further reduces the cost and boosts cell product quality.

### BRIEF DESCRIPTION OF THE DRAWINGS

FIG. 1A shows a side view of a PLAX system for parallel multi-sample centrifugation using a swinging bucket mechanism.

FIG. 1B shows a top view of the rotor/cassette subsystem of a PLAX system for parallel multi-sample centrifugation using a swinging bucket mechanism.

FIG. 2A shows a cross-sectional view of a swinging bucket cassette housing a processing chamber and tubing.

FIG. 2B shows a front view of a processing chamber with connected tubing.

FIG. 3A shows a top view of an empty foldable swinging bucket cassette in an open configuration.

FIG. 3B shows a side perspective view of the foldable swinging bucket cassette in a closed configuration.

FIG. 4A shows two exemplary embodiments of contents of processing chambers configured to be oppositely positioned on the rotor.

FIG. 4B shows exemplary centrifugation in two different directions with respective processing chamber configurations shown in FIG. 4A.

FIG. 5 shows an application of the PLAX system wherein microbubbles are used for cell isolation by processing microbubble bound cells.

FIG. 6 shows a mixing mechanism in connection with the rotor/cassette subsystem of the PLAX system.

FIG. 7A shows a partial exploded perspective view of the rotor/cassette subsystem including a rotor and swinging bucket cassette.

FIG. 7B shows a top perspective view of the rotor/cassette subsystem including two cassettes.

FIG. 7C show a front view of the rotor/cassette subsystem of FIG. 7B with two cassettes in a vertical orientation.

FIG. 7D show a front view of the rotor/cassette subsystem of FIG. 7B with two cassettes in a horizontal orientation.

FIG. 8A shows a front view of a PLAX hydraulic pump subsystem.

FIG. 8B shows a top perspective view of a PLAX hydraulic pump subsystem.

FIG. 9 shows a schematic of a PLAX pre-mixing subsystem.

FIG. 10 shows a schematic of a PLAX cabinet and systematization containing instrumentation and devices for controlling processing.

FIG. 11A shows a schematic of a PLAX in-line monitoring subsystem.

FIG. 11B shows a close-up view of connectors of the PLAX in-line monitoring subsystem.

FIG. 12 is a schematic of a PLAX system according to an embodiment of the present disclosure.

FIG. 13A shows the priming process step of the embodiment shown in FIG. 12.

FIG. 13B shows the blood-in process step of the embodiment shown in FIG. 12.

FIG. 13C shows the density media-in process step of the embodiment shown in FIG. 12.

FIG. 13D shows the centrifugation process step of the embodiment shown in FIG. 12.

FIG. 13E shows the erythrocyte collection process step of the embodiment shown in FIG. 12.

FIG. 13F shows the density gradient media extraction process step of the embodiment shown in FIG. 12.

FIG. 13G shows the rotated-cassette plasma extraction process step of the embodiment shown in FIG. 12.

FIG. 13H shows the rotated-cassette mononuclear cell retention process step of the embodiment shown in FIG. 12.

FIG. 13I shows the mononuclear cell extraction process step of the embodiment shown in FIG. 12.

FIG. 13J shows the plasma extraction process step of the embodiment shown in FIG. 12.

FIG. 14A shows a bar plot graphing accuracy over pumping steps for reagents in for a hydraulic pump.

FIG. 14B shows a bar plot graphing accuracy over pumping steps for reagents out for a hydraulic pump.

FIG. 14C shows a bar plot graphing accuracy over pumping steps for reagents towards cassette for a peristaltic pump.

FIG. 14D shows a bar plot graphing accuracy over pumping steps for flush PBS/washing for a peristaltic pump.

FIG. 14E shows a comparison of the accuracy of the respective plots in FIGS. 14A-14D.

FIG. 15A shows a hyperbolic pressurization plot.

FIG. 15B shows a linear pressurization plot.

FIG. 15C shows a stepwise pressurization plot.

FIG. 15D shows a hyperbolic depressurization plot.

FIG. 15E shows a linear depressurization plot.

FIG. 15F shows a stepwise depressurization plot.

### DETAILED DESCRIPTION OF THE DRAWINGS

The following description and drawings are illustrative and are not to be construed as limiting. Numerous specific details are described to provide a thorough understanding. However, in certain instances, well known or conventional details are not described in order to avoid obscuring the description. References to one or an embodiment in the present disclosure are not necessarily references to the same embodiment, and such references mean at least one.

The use of headings herein is merely provided for ease of reference and shall not be interpreted in any way to limit this disclosure or the following claims.

Reference in this specification to “one embodiment” or “an embodiment” or the like means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described that may be exhibited by some embodiments and not by others. Similarly, various requirements are described that may be requirements for some embodiments but not other embodiments. The bolded labels in the figure descriptions shall have the meanings ascribed to them in the drawings themselves.

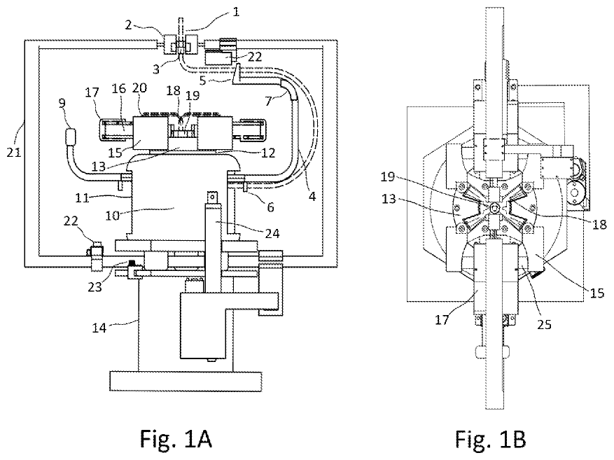

FIG. 1A shows a side view of the PLAX system that can perform parallel multi-sample centrifugation in fashion familiar to biomedical researchers and staff. FIG. 1A specifically focuses on a swinging bucket mechanism. A bundle of flexible medical grade tubes 1 is located at the upper extremity of the PLAX system. The bundle of tubes 1 interface with a spring adapter 2, which is attached to a tube locking input 3 that ensures that the tubing 1 remains stationary and tight during the entire separation process. The central tubing 1, which may be made of biocompatible materials, is curvedly routed through an arm housing 4, secured via a ring enclosed structure input 5 and ring enclosed structure output 6 having stoppers 7 to prevent the tubing 1 from interfering with the centrifugation/separation process. The curved arm housing 4 is counterweighted with a counterweight structure 9 with similar weight in the opposite direction of the system to promote smooth operation and balance under centrifugation. In certain embodiments, an additional arm 15 may be provided as a counterweight. Arm housing 4 and counterweight 9 are mechanically fixed to a seal-less anti-twisting centrifuge 10, therefore rotating during centrifugation. The core and gear of the centrifuge 10 include a central centrifuge housing 11 formed of material that allows its secure operation and isolation from the rest of the system. A rotational element (e.g., ball bearing) 12 is mounted on a mechanical fixing, namely a central bush, fitted in the upper extremity of the centrifuge 10, while the rotor 13 is attached to the central bush ensuring proper alignment when inserted/positioned into the centrifuge rotor 13. When utilizing a gear set configured as outlined in one of U.S. Patent Publication Nos. U.S. Pat. Nos. 3,586,413 A, 4,425,112 A, 4,900,298 A, the rotational speed ratio between rotor 13 and centrifuge 10 is precisely 2:1 upon activation of motor 14. Arms 15 are placed onto the rotor 13 to allow for the positioning of a swinging bucket cassette 16 that includes a locking mechanism 17. The swinging bucket cassette 16 houses a processing chamber 26. The bundle of tubes 1 is routed towards the inside of the central centrifuge housing 11 in a loop-like fashion, and is further attached to a tube locking output 19. Maintaining the bundle of flexible tubes 1 secured and fixed in both the upper and lower extremities of the centrifuge 10 via a respective tube locking input 3 and tube locking output 19, prevents twisting of the tubes 1 under centrifugation by routing the tubes 1 in a “U” shape. Once the bundle of flexible tubes 1 is redirected to the central centrifuge housing 11, a tube diverter 18 allows the re-routing of each of these tubing lines through the cassette 16 and towards the corresponding inlets and outlets of the processing chamber 26, via retaining channels 20 on the arms 15 and rotor 13. Optionally, a frame 21 may be located in the periphery of the centrifuge 10 to allow for the placement of distinct sensors and devices in order to ensure a smooth processing operation and system performance. The PLAX frame 21 may be equipped with ultrasonic sensors 22 that allow the centrifuge rotor 13 to be situated in a fixed position and to align the swinging bucket cassette 16 with the mixing device 24. In a preferred embodiment, a built-in triple axis accelerometer 23 may be integrated within the PLAX system as a safety feature, allowing the monitoring and stabilization of the system for imbalances due to any abrupt, sudden, or unexpected external factor.

FIG. 1B shows a top view of the described PLAX system, specifically the rotor/cassette subsystem. In a preferred embodiment, the swinging bucket cassettes 16 may be inserted/placed in the central centrifuge rotor 13 via incorporated rotor bucket supports 25, allowing the swinging bucket cassettes 16 to remain seated at a resting vertical position. As stated, rotor 13 may be engaged and driven via a single motor 14. Arms 15 are placed onto the rotor 13 to allow for the positioning of a swinging bucket cassette 16 and engagement via locking mechanism 17. A tube diverter 18 may be placed proximate to the central axis of rotor 13 to facilitate the re-routing of respective tubing lines through the cassette 16.

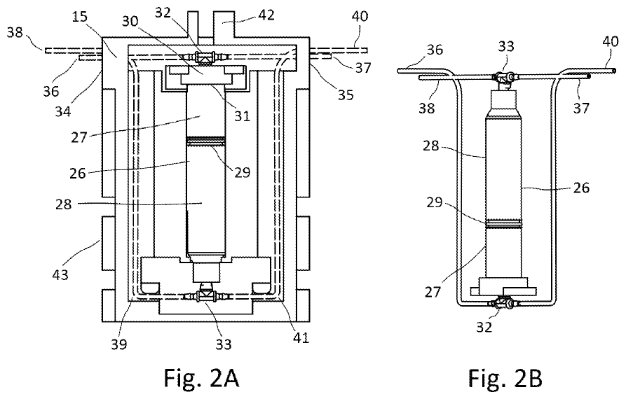

FIG. 2A shows an inside view of a PLAX swinging bucket cassette 16, which holds a processing chamber 26. The processing chamber 26 may be characterized as a syringe-like isolated container comprising an upper body 27 and a lower body 28. A piston 29 is disposed between the upper body 27 and lower body 28. The processing chamber 26 is sealed with a cap 30 and equipped with an O-ring 31 in the upper body 27 of the processing chamber 26, thus ensuring airtightness between the upper section of the processing chamber 26 and the piston 29. In a preferred embodiment, the processing chamber 26 is made of a medical grade transparent material such as polycarbonate. For processing biological samples, the PLAX system uses the piston 29 to maintain a closed continuous system. Therefore, samples within the medical grade syringe barrel processing chamber 26 can be moved in or out anytime, even during centrifugation, due to the incorporation of a plurality of inlet cavities 34 and outlet cavities 35 within the swinging bucket cassette 16.

The upper body 27 of the processing chamber 26 includes a tubing connector 32 for hydraulic tubing 36 that allows the piston 29 to move from top to bottom within the syringe barrel processing chamber 26, thereby facilitating precise movement of fluid in and out of the processing chamber 26. Likewise, a tubing connector 33 is included at the lower body 28 of the processing chamber 26.

Input tubing 38 (comprising part of bundle of tubes 1) is connected to the pre-mixing subsystem 124 and buffer bag 113, thus routed via incorporated input retaining channels 39 within the cassette 16, where the input retaining channels 39 are configured to allow for tubing placement and positioning. Similarly, the resultant diverse factions outputted from the centrifugation process are redirected towards their corresponding waste bag 95 or fraction collection container 96, 97, 98 via output tubing 40. Outlet tubing 40 is routed similarly via its analogous output retaining channels 41.

FIG. 2B shows a view of the processing chamber 26 and related tubing connections independent of the housing. Additionally, in the embodiment shown in FIG. 2B, the processing chamber 26 is oriented in a second configuration, such that the lower body 28 of the processing chamber 26 may be proximate to the hinge 42. As is apparent, the swinging bucket cassette 16 may be configured to house the processing chamber 26 and associated tubing and connections regardless of whether the processing chamber 26 is arranged in the first or second orientation.

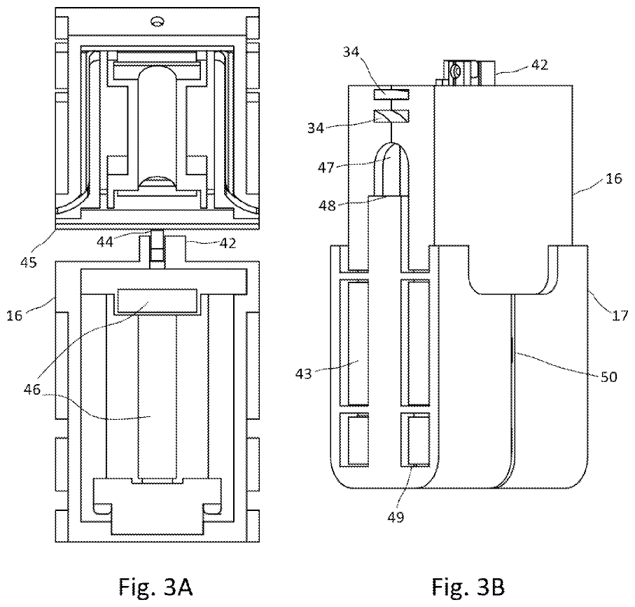

FIG. 3A shows the folding housing in an open configuration without the processing chamber 26 inserted into it. As can be seen, the inside of the cassette 16 features input retaining channels 39 and output retaining channels 41 to accommodate tubing, a channel for a hydraulic pump 123, as well as a pocket 46 for housing the processing chamber 26. The cassette 16 may include a hinge mechanism 42 to facilitate easy foldability, allowing it to get into close contact with its reciprocal cassette 45. As such, the swinging bucket cassette 16 is detachable and openable for easy installation and connection between the processing chamber 26 and the single use set/bundle of tubes 1. According to an embodiment of the swinging bucket cassette 16, the cassette 16 may be designed with a pocket 46, specifically tailored to fit the processing chamber 26 in two orientations depending on user processing preferences. More specifically, in a first orientation the upper body 27 of processing chamber 26 may be proximate to the hinge 42, while in a second orientation the lower body 28 of the processing chamber 26 may be proximate to the hinge 42.

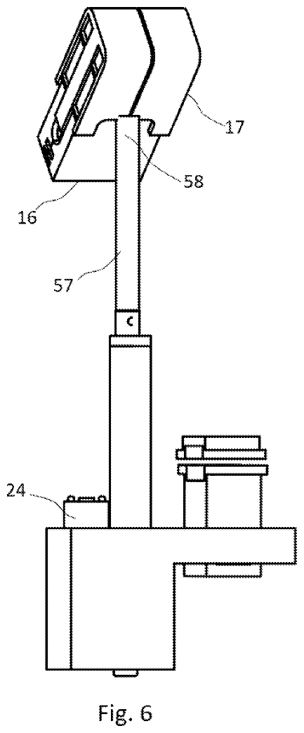

FIG. 3B shows the outside of the housing when the foldable cassette 16 is in a closed configuration. Upon closing the foldable cassette 16 at hinge 42, both symmetrical or non-symmetrical shaped parts of the swinging bucket cassette 16 form a specific pattern 43 that mirrors its cassette counterpart pattern 49, therefore allowing the respective sides of the cassette 16 to remain fixed and closed with a cassette locker 17 prior processing or centrifugation. Such cassette locker 17 has a lock and release latch mechanism 50 that grants ease of use by enabling effortless opening and closing. Additionally, the cassette locker 17 facilitates the formation of a physical support 48 for rotation and mixing purposes by the mixing device 24 (shown in FIG. 6) forming a bushing 47, when getting placed in the central rotor 13 of the PLAX system, and when a given sample is under an incubation or washing protocol.

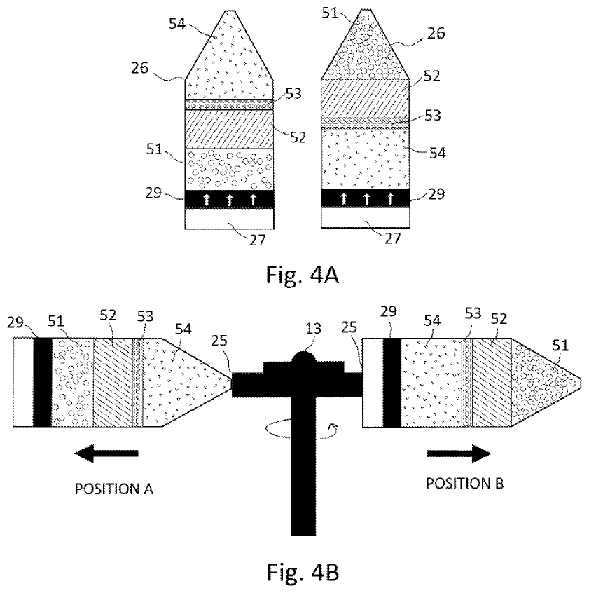

FIGS. 4, 5, and 13 show diagrams applications of the present disclosure for novel microbubble-based cell separation, activation, expansion, and transduction (Patent U.S. Pat. No. 10,479,976B2, US20220154150A1) and convenient integration into other cell processing workflows, such as density based gradient separation (DGBS), to accomplish automation without human intervention.

FIG. 4A shows two different embodiments of the contents of processing chambers 26 configured to be positioned on the rotor 13 in opposite directions. The processing chamber 26 includes an upper body 27 and the lower body 28 having a tapered end. Inside the processing chamber 26 shown on the left, the piston 29 is arranged proximate to the end of the upper body 27. During operation, the piston 29 is urged towards the lower body 28. Erythrocytes 51 are provided closest to the piston 29, followed by a density separation medium 52, mononuclear cells 53, and plasma 54 proximate to the lower body 28. Inside the processing chamber 26 shown on the right, the piston 29 is also arranged proximate to the end of the upper body 27, and the piston 29 similarly is urged towards the lower body 28. However, in this configuration, plasma 54 is provided closest to the piston 29, followed by mononuclear cells 53, a density separation medium 52, and erythrocytes 51 proximate to the lower body 28.

FIG. 4B shows exemplary centrifugation in two different directions with the respective processing chamber 26 content configurations shown in FIG. 4A, which may be switched alternatively, as enabled by the design of the swinging bucket cassette 16.

Rotor 13 is arranged in an upright configuration, and rotates about an axis. One or more rotor bucket supports 25 are provided on the rotor 13 to allow for attachment of the swinging bucket cassettes 16. Swinging bucket cassettes 16 can get inserted/placed in the central centrifuge rotor 13 due to the incorporation of rotor bucket supports 25, thus allowing it to remain seated, at a resting vertical position.

For example, by spinning in position B, erythrocytes 51 and the density separation medium 52 can be removed, while maintaining mononuclear cells 53 and plasma 54 within the processing chamber 26. Subsequently, by switching to position A, mononuclear cells 53 can be retained and washed in the processing chamber 26, while first extracting the plasma 54. Therefore, the washed mononuclear cells 53 can be used for subsequent procedures, such as targeted microbubble-based cell isolation 56, T cell activation, transduction, and short-term cell culture, without needing to leave the processing chamber 26. (U.S. Pat. No. 10,479,976B2, US20220154150A1)

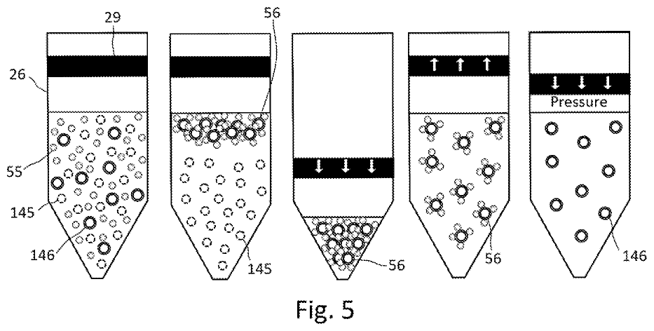

FIG. 5 shows an application of PLAX system wherein microbubbles 55 are used for cell isolation by processing microbubble bound cells. This allows for specific and relatively easy separation from the rest of non-targeted cells 145, as the bound cells get separated and the others tend to sink, even without centrifugation.

The piston 29 contained within the processing chamber 26 can move from the upper body 27 to the lower body 28 of the processing chamber 26. This is enabled by its tubing 36 in connection with a hydraulic pump 123. As the hydraulic pump 123 moves liquid out through the hydraulic tubing 36, the piston 29 within the processing chamber 26 moves down moving liquid out of the processing chamber 26, and vice versa.

First, the PLAX system is subject to a priming process from the hydraulic tubing 36 to the degassing chamber 118, followed by the transfer of blood from the mixing system 124, or blood bag 141 into the processing chamber 26. The blood may contain erythrocytes 51, mononuclear cells 53, and plasma 54.

Subsequently, microbubbles 55 are introduced into the processing chamber 26, and the swinging bucket cassette 16 is mixed by the mixing device 24 for a pre-set period of time in accordance with a given protocol. After interaction and centrifugation, bound cells (e.g., microbubbles 55 attached to targeted cells 146) are separated from non-targeted cells 145 to the low-gravity zone, while the rest of non-targeted cells 145 are extracted from the processing chamber 26.

Remarkably, the lipid shelled microbubbles 55 can undergo dissolution in 1-2 days spontaneously, or immediately by increasing ambient pressure as described in U.S. Patent U.S. Pat. No. 10,479,976 B2 and U.S. Patent Application Publication US 2022/0154150. This is a very useful property for streamlining the cell processing workflow and easing engineering complexity for automation.

Microbubbles 55 bound to target cells 146 can be quickly disrupted by increasing ambient pressure to about 2 atm by moving the piston 29 with the hydraulic pump 123 and increasing the internal pressure within the processing chamber 26. A greater or lesser ambient pressure may be set depending on the concentration. Increasing the internal pressure for microbubble 55 disruption may be achieved not only by moving the piston 29 downwards, but also with the use of an air compressor connected to the upper body 27 or lower body 28 of the processing chamber 26, or with the use of hydrostatic pressure.

FIG. 6 shows the PLAX cassette/chamber subsystem mixing mechanism. The mixing mechanism includes the mixing device 24 disposed near the base of the structure. A moving/extending/reaching structure 57 extends upwards from the base, and may be adjusted in translational motion to move the swinging bucket cassette 16 disposed at the distal end of the moving/extending/reaching structure 57. The moving/extending/reaching structure 57 may connect to the cassette bottom 58 of the swinging bucket cassette 16. The moving/extending/reaching structure 57 can for instance include a telescoping arm mechanism.

When the centrifuge 10 stops, mixing device 24 may continue to facilitate mixing of the samples (e.g., microbubbles 55 and target cells 146) by moving the cassette bottom 58. The mixing device 24 may comprise any combination of linear actuators, pneumatic systems, motors, and robotic arms. The point of contact may be physical, magnetic, a lever, etc.

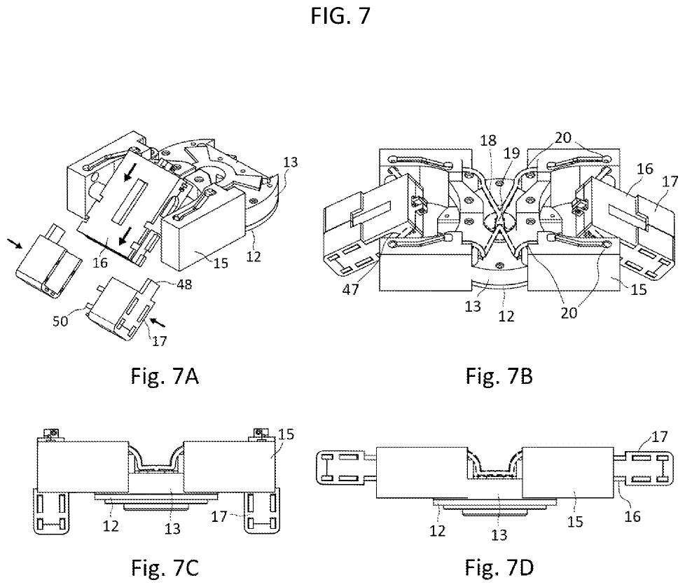

FIGS. 7A-7D show various views of the rotor/cassette subsystem. The figures demonstrate the range of motion of the swinging bucket cassette 16, and its relationship with the rotor 13, rotational element 12, and associated tube channels. While two cassettes 16 are depicted in these figures, additional cassettes 16 can be symmetrically installed.

FIG. 7A shows an exploded perspective view of the rotor/cassette subsystem that includes the rotor 13 and swinging bucket cassette 16. The assembly includes rotor 13 disposed atop a rotational element 12 to ensure proper alignment when positioned on centrifuge 10. Arms 15 extend outwards from the rotor 13, and include intermediate space to permit the cassette 16 to be rotatably attached between respective arms 15. The cassette 16 may be secured by a cassette locker 17 having a physical support 48 and a lock and release latch mechanism 50 that enables effortless opening and closing.

FIG. 7B shows the cassette locker 17 with lock and release latch mechanism 50 interfacing with the respective cassettes 16 that are disposed on opposite sides of rotor 13 in a tilted orientation. The depicted embodiment is shown with tubing and related elements for routing the tubing through the central axis of rotor 13. A tube diverter 18 re-routes each of these tubing lines 1 towards the cassettes 16 through retaining channels 20 included in the arms 15, with attachment to tube locking output 19. In a preferred embodiment, bushing 47 may be included at the connection points between arms 15 and cassette 16 to reduce friction.

FIG. 7C and FIG. 7D show side views of the assembly, wherein the cassettes 16 are oriented vertically and horizontally, respectively.

Upon the positioning of cassettes 16, they are closed, secured, and locked by the attachment of the cassette lockers 17. When in a resting position, the cassettes 16 are positioned in a vertical orientation aligned parallel to the centrifuge 10 gears and centrifuge housing 11 (seen in FIG. 1A), as is typical for a swinging bucket centrifuge system. As centrifugation ramps up in speed, the swinging bucket cassettes 16 transition from the vertical resting position shown in FIG. 7C to a horizontal positioning shown in FIG. 7D, being dragged by the outward g-force exerted by the speed of rotation. While rotating in the horizontal orientation during centrifugation, particle separation results.

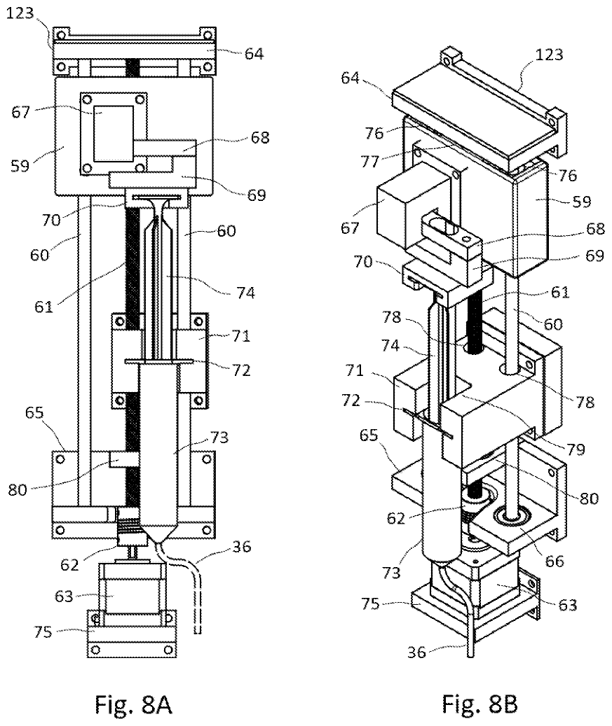

FIGS. 8A and 8B respectively show a front view and a top perspective view of a PLAX hydraulic pump subsystem comprising the hydraulic pump 123 that controls positioning and fluidics for piston 29.

The hydraulic pump 123 is a plunger control subsystem that employs a stepper motor 63 coupled to a leadscrew 61 via a shaft coupling connector 62 to drive a plunger-flange 70 of a processing buffer container 73 (e.g., medical syringe barrel). Hydraulic tubing 36 may extend outwards from the bottom of processing buffer container 73. Sensors such as weight cells 68 may be integrated into the subsystem to allow for feedback control and accurate volume transfer during piston 29 translation within the processing chamber 26. The hydraulic pump 123 subsystem comprises a movable pressurization platform 59 that moves along the length of the leadscrew 61 via a leadscrew translation adaptor 77 (e.g., leadscrew nut). The movable pressurization platform 59 includes corresponding sensor cavity-coupling integrations 67 to which weight cells 68 may attach. A syringe attachment 69 may be disposed between weight cells 68 and flange 70 on the movable pressurization platform 59.

Linear rail shaft optical axis rod 60 and multiple rotational elements 66, 76 (e.g., ball bearings) may be provided to facilitate translation of the dynamic pressurization platform 59. Driving the flange 70 with proper stability may be achieved by providing any combination of a top support structure 64, a bottom support structure 65, and a stepper motor support structure 75. The stepper motor support structure 75 may be arranged proximate to the stepper motor 63. The top support structure 64 may be included at the top of the hydraulic pump 123, and the bottom support structure 65 may be included proximate to the shaft coupling connector 62. A holder 71 may secure the hydraulic processing buffer container 73 through the barrel flange 72 within the PLAX hydraulic pump subsystem. Holder 71 may include a plurality of holes 78, 79 and indentations 80 for placement of the processing buffer container 73 and related elements, in a manner that allows both adjustability and tight grip, thereby allowing free translation of the plunger 74 and motion of the flange 70. The hydraulic pump 123 and one peristaltic pump 108 may be installed in the system to move fluid in and out of the syringe-like processing chamber 26. Through the described features, the hydraulic pump 123 system enables accurate fluid volume control, from either a stationary container (e.g., blood sample, washing buffer) or from a pre-mixing subsystem 124, towards feeding sample and reagents, to a rotating processing chamber 26 and extracting fractions into stationary containers 96, 97 and 98. The use of peristaltic pump 108 may additionally be employed to completely transition cell fractions towards stationary containers 96, 97 and 98 and waste to waste bag 95.

In a preferred embodiment, a sterile flexible cover/enclosure may be applied to the upper part of the hydraulic pump syringe 73 comprising plunger 74, barrel flange 72, and plunger flange 70, to shield contaminants from the environment, as described in U.S. Patent Publication U.S. Pat. No. 4,713,060 A.

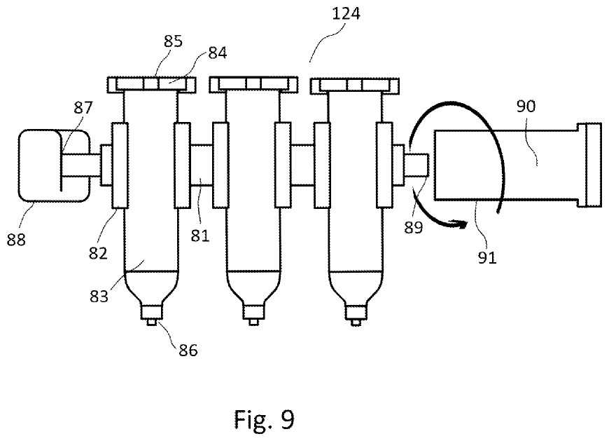

FIG. 9 shows a schematic of a PLAX pre-mixing subsystem 124. All reagents (e.g., microbubbles 55, target cells 146, density gradient media 52 or blood) that are required to be incorporated within the processing chamber 26 prior to processing are held in a pre-mixing subsystem 124 and connected via a medical grade input tubing 38. The PLAX pre-mixing subsystem 124 comprises a stepper motor 90 within a housing 91, coupled to a rod mixer 81 via a coupling connector/adapter 89. The rod mixer 81 connects to a support structure 88 at the end opposite of the coupling connector adaptor 89. The support structure 88 includes rotational elements 87 (e.g., ball bearings) that reduce rotational friction and support axial and radial loads. The rod mixer 81 may be configured to hold and secure multiple input reagent containers 83 (e.g., syringes) simultaneously via a clamp mechanism 82 that allows easy placement and removal when required. The input reagent containers 83 are sealed via a sealing cap 84, coupled with a filter 85 (e.g., 0.2 μm filter or membrane) and individually connected to the rest of the system via a tubing connector 86. Specifically, when dealing with large volumes such as blood, the reagents 53, 55 could be contained within the confines of medical grade bags in the pre-mixing subsystem 124. Depending on the type of reagents, specifically when dealing with cell isolation by using microbubbles 55 or density gradient media 52 it is important to have a homogeneous solution prior its introduction to the processing chamber 26 to preserve an accurate concentration. The PLAX pre-mixing subsystem 124 has the capability of ensuring homogeneous reagent consistency by mixing and rotating the containers 83 (e.g., 180 deg) for a pre-set amount of time prior to loading within the processing chamber 26 when moving upwards piston 29 and actuating hydraulic pump 123. The complete use of the pre-mixing system 124 will vary depending on the protocol as some features may not be employed (system may have additional clamps 82, for the use of additional containers 83).

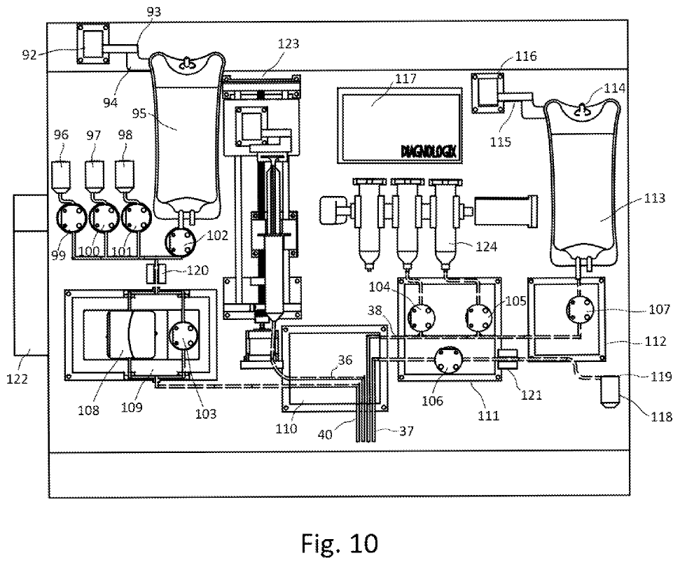

FIG. 10 shows a schematic of a PLAX cabinet and systematization containing instrumentation and devices for controlling the processing. The cabinet is the hub for the subsystems detailed in FIG. 8 and FIG. 9, namely hydraulic pump 123 subsystem and reagent/input pre-mixing system 124, as well as the holder of medical components and devices necessary for proper fluidic transport within the system.

The cabinet contains a washing buffer bag 113 configured to hold a liquid such as phosphate buffered saline, cell culture media, etc. The tubing extends from the washing buffer bag 113 to the input tubing line 38 of cassette 16, with a buffer pinch valve 107 disposed intermediate the washing buffer bag 113 and cassette 16. The washing buffer bag 113 tubing passes by the output tubing of pre-mixing system 124, which may be sealed by valves 104, 105. The bundle of tubes 1 that respectively are routed curvedly through arm housing 4, towards centrifuge 10 and processing chamber 26 may include input tubing 36, 38, and output tubing 37, 40.

A waste collection bag 95 is coupled to a pre-collection peristaltic pump 108, to route, re-direct and move liquid out of the processing chamber 26. Buffer bag 113 is the responsible reservoir for holding buffers used in subsequent washing processes/protocols, whereas the waste bag 95 is responsible for the removal of unwanted secondary fractions or substances through a waste pinch valve 102. Buffer bag 113 is connected to a sensor 115 (e.g., weight sensor) housed in housing 116, and is attached to the cabinet via hook connector 114. Similarly, waste bag 95 is connected to a sensor 93 (e.g., weight sensor) housed in housing 92, and is attached to the cabinet via hook connector 94. Sensors 93, 115 provide real-time data, tracking the processed and employed volume across the system from input to output. Fractions of interest can be collected in bags/containers 96, 97, 98 using individual or multichannel external pinch valves 99, 100, 101, respectively. This prevents leakage and contamination in adjacent lines, and prevents interference with the fluid exiting the processing chamber 26 through a pre-collection pinch valve 103. Similarly, reagents such as microbubbles 55 and target cells 53 may move out from the mixing sub-system 124 towards the processing chamber 26 via the medical grade tubing 1 by opening corresponding pinch valves 104, 105, which may individually correlate to an input reagent container 83 (shown in FIG. 9). Alternatively, the tubing may be multichannel.

An optical sensor 120 allows for sensing of colors in the medical grade tubing 1, therefore enabling detection of the distinct fractions exiting the processing chamber 26. The optical sensor 120 may be included between the chamber/rotor subsystem and the plurality of containers 95, 96, 97, 98.

The cabinet architecture may optionally incorporate retaining channels 110 for the tubing connections, or housing adapters 109, 111, 112 for various system components, such as the peristaltic pump 108, degassing pinch valve 106, and buffer pinch valve 107, respectively.

Any remaining air within the tubing-set for priming purposes is routed through a degassing pinch valve 106 towards a degassing chamber 118, which is coupled to a filter 119 (e.g., 0.2 μm filter or membrane). Reagents such as microbubbles 55, target cells 53, or density gradient media 52 are incorporated into the processing chamber 26 via input tubing 38 in the lower body 28 portion of the processing chamber 26.

Additionally, the PLAX system may include a tube welding subsystem 122 for sealing and closing tubing connected to the processing chamber 26 within the swinging bucket cassette 16. While in operation, this device can execute the continuous and accurate movement of liquid in and out of system via the incorporation of an intuitive user interface screen 117, which may optionally have touch responsive capabilities. The fraction of interest may be cultured within the processing chamber 26 for a short-term period, such as a few days, to allow for completion of the streamlined process of cell sorting, activation, and transduction, prior to transferring the engineered cells to a separate culture bag for longer term cell culture. Such culture bags are widely available commercially. Culture media and CO₂ gas can be delivered and exchanged by connecting further tubing lines in addition to the ones currently used to transfer materials into the processing chamber 26 via input tubing 38 and out via output tubing 40. In certain embodiments, a “water jacket” may be used within the cassette 16 for short cell culture. When needed, the water jacket may be connected to an external pump in the PLAX cabinet that circulates water of desired temperature.

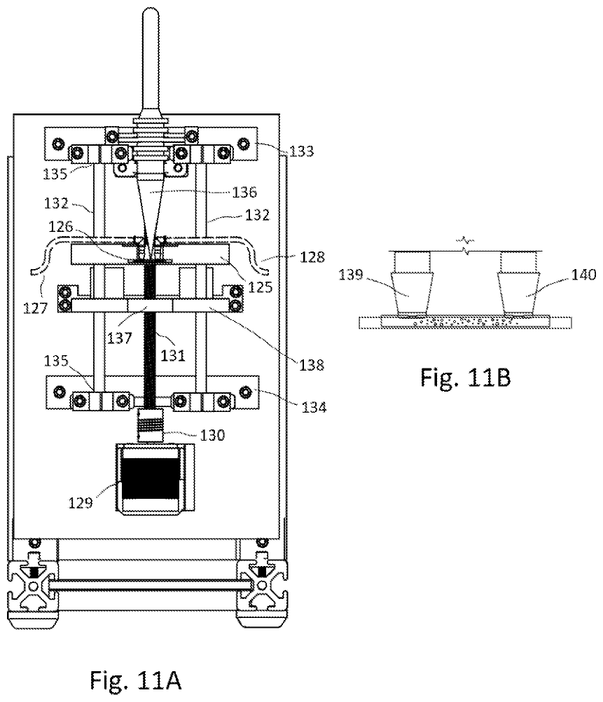

FIG. 11A shows a schematic of the PLAX in-line monitoring subsystem. Standard and specific in-line monitoring modules are implemented within the PLAX system, and may optionally be employed within certain processes for gathering live-on-demand accurate and reliable data within the system. Exemplary monitored parameters include cell morphology, temperature, concentration, and size distribution. This inline-monitoring subsystem comprises a free moving dynamic focusing platform 125 configured to autofocus a microfluidic chamber 126. The microfluidic chamber 126 has an inlet 127 and an outlet 128, via a connector 139, 140 respectively, holding the to-be-analyzed fraction or sample of interest within an enclosed channel that allows for precise control of fluid flow and manipulation of e.g., microbubbles 55 or target cells 53 contained within it. The dynamic platform 125 may move up and down freely, as needed, when focusing and moving though a lead screw 131, which provides guidance. For calibration and focusing purposes, a stepper motor 129 connected to a shaft coupling connector 130 may move the microfluidic chamber 126, which may be reinforced with linear motion axis rail 132 and supported by rail clamps 135. The monitoring sub-system may optionally incorporate a top support structure 133, middle support structure 138 (which may include a plurality of holes), and bottom support structure 134, to improve stability. The imaging device 137 is attached parallel to the platform 125 a light source 136 (e.g., LED, infrared, fluorescence or coupled with filters) providing appropriate illumination. The beam of light from light source 136 crosses the transparent microfluidic chamber 126 and the exposed pixels of the sample allow for high resolution (μm×pixel) imaging of the sample. Data collection by the in-line monitoring sub-system can be processed to analyze the size, concentration, and shape of the samples (e.g., microbubbles 55 and target cells 53) in real-time. These data parameters may be complied as a result of accurate microscope control, image capture, image processing/analysis, including image filtering, noise reduction, segmentation, and detection. Through these features, the system reduces the error inherent to manual sampling with traditional microscopes and hemacytometers, therefore ensuring cell viability and integrity prior cell culture. Observing in real-time the physical characteristics of cells, including their shape, size, identification of abnormalities or changes, is particularly useful for early detection/diagnosis of engineered cell products.

FIG. 11B shows a close-up view of connectors 139, 140 of the PLAX in-line monitoring subsystem. Connector 139 corresponds to microfluidic chamber input 127, while connector 140 corresponds to microfluidic chamber output 128.

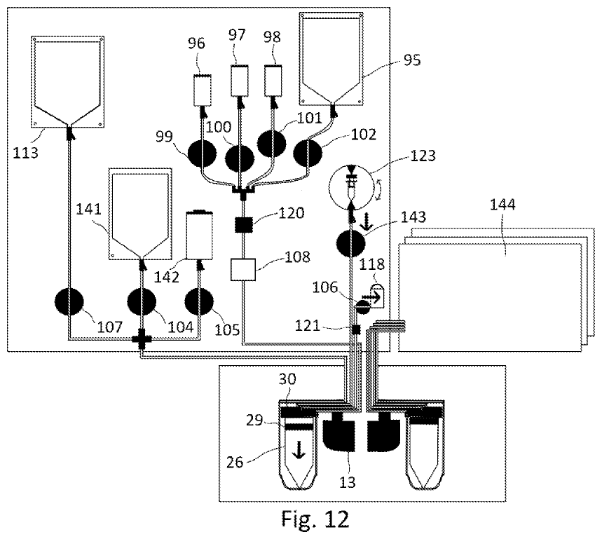

FIG. 12 shows a PLAX system with instrumentation, subsystems, and devices for controlling processing. The rotor/cassette subsystem of the PLAX system includes the rotor 13 with at least one attached processing chamber 26 containing a piston 29. The processing chamber 26 is sealed by a cap 30. In the shown embodiment, two processing chambers 26 are included. A plurality of tubes connects the rotor/cassette subsystem to the other constituent system elements.

The rotor/cassette subsystem is connected to hydraulic pump 123 via tubing with an intermediate pinch valve 143. Another line of tubing connects the rotor/cassette subsystem to a degassing chamber 118 with an intermediate degassing pinch valve 106. A bubble air trapping detection and pressure monitoring sensor 121 may be included between the rotor/cassette subsystem and the degassing pinch valve 106. Tubing may connect the rotor/cassette subsystem to a set of parallel control/electronic module 144. For multiplexing processing, additional bundles of tubes can be simultaneously installed alongside the first sets of tubing using multi-channel mechanisms, including multiple-channel pinch valves and peristaltic pumps 108.

A further line of tubing connects the rotor/cassette subsystem to waste bag 95 and fraction collection containers 96, 97, 98. Each respective fraction collection container 96, 97, 98 includes a corresponding pinch valve 99, 100, 101 that controls the respective channel, while waste bag 95 corresponds to pinch valve 102. A pre-collection peristaltic pump 108 and an optical sensor 120 may be disposed intermediate the rotor/cassette subsystem and fraction collection containers 95, 96, 97, 98.

The rotor/cassette subsystem is connected to a plurality of bags, namely buffer bag 113, blood bag 141, and density gradient separation media bag 142 via respective tubing lines. Each bag 113, 141, 142 has an individual tubing line and a respective pinch valve 107, 104, 105. Blood bag 141 contains the blood input, and the tubing line is sterilely connected to the lower body 28 of the processing chamber 26 via a sterile connecting device.

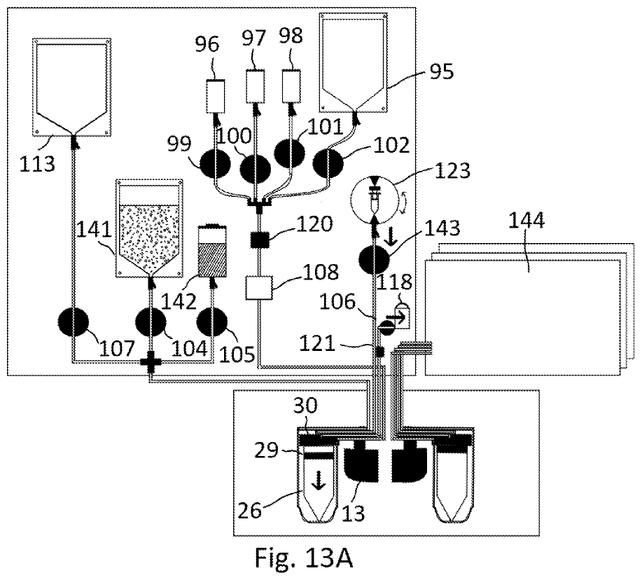

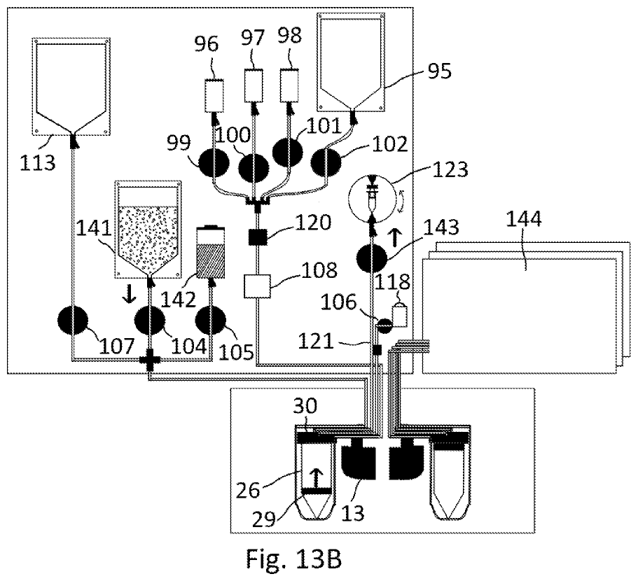

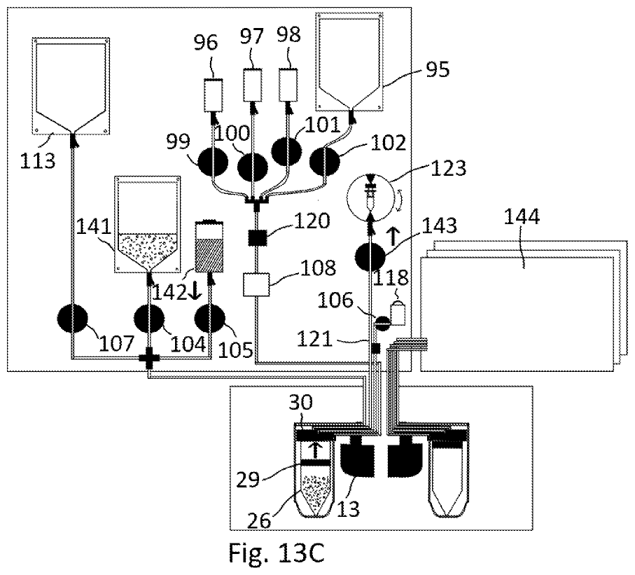

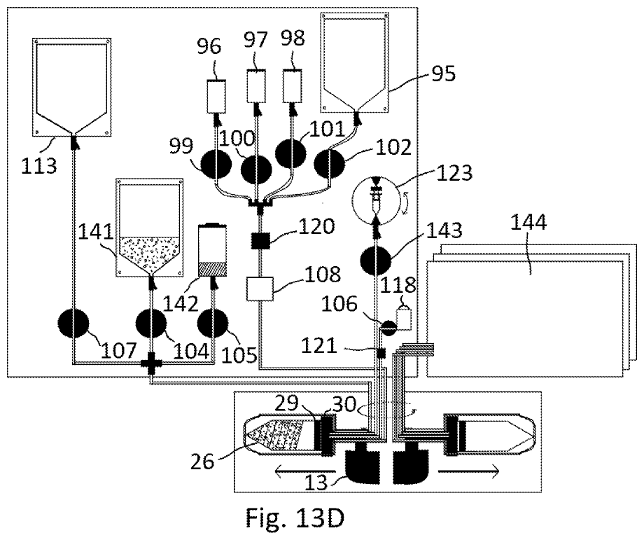

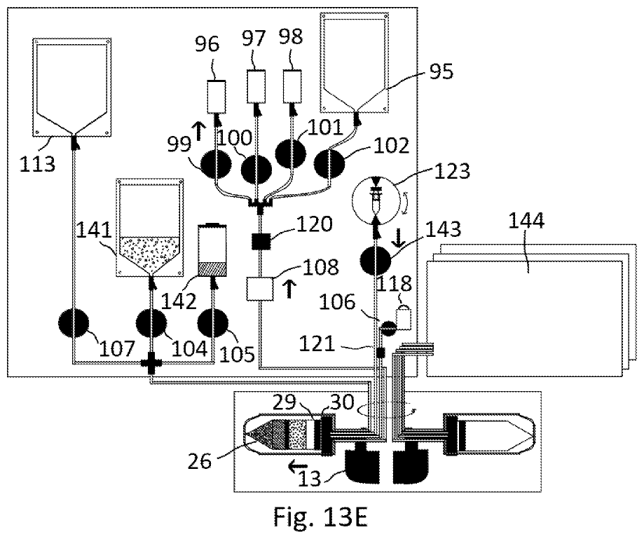

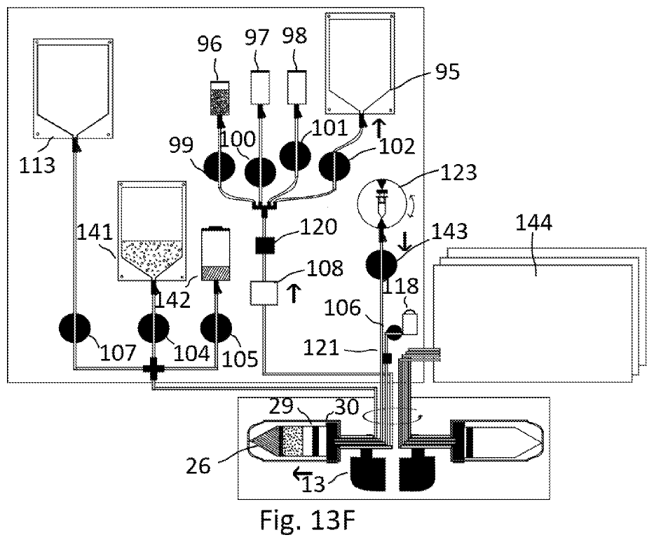

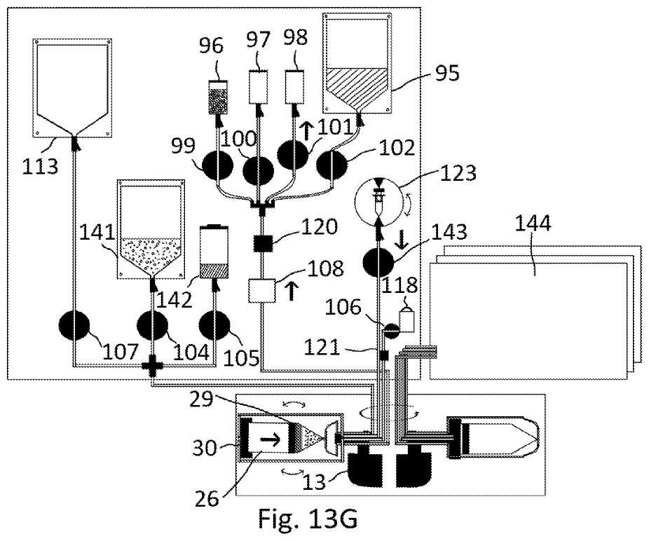

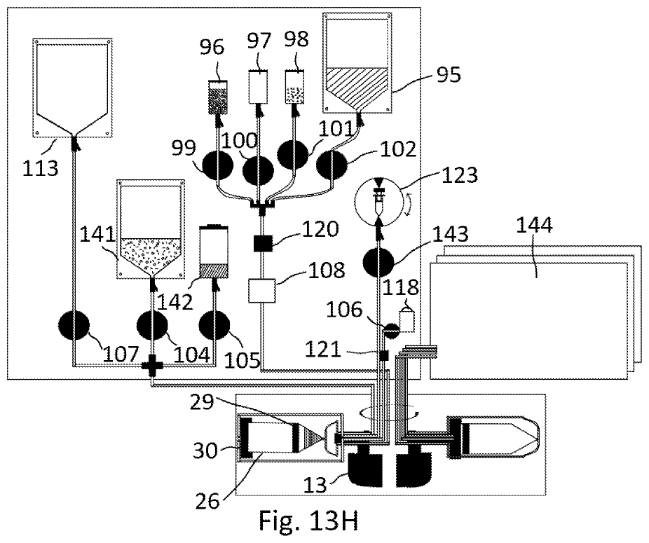

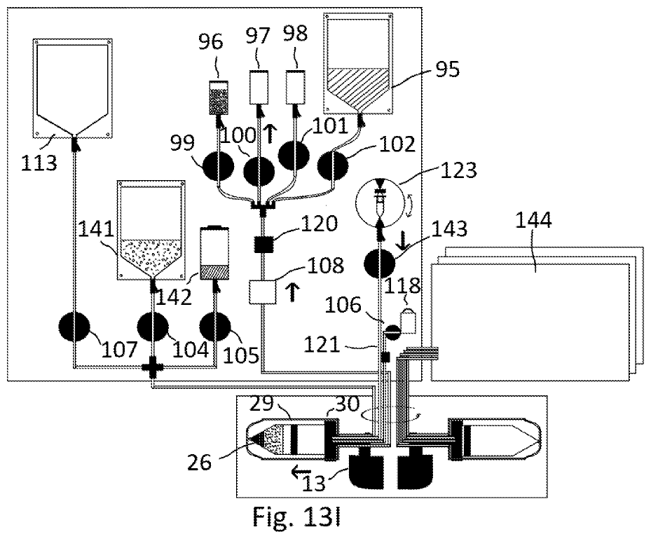

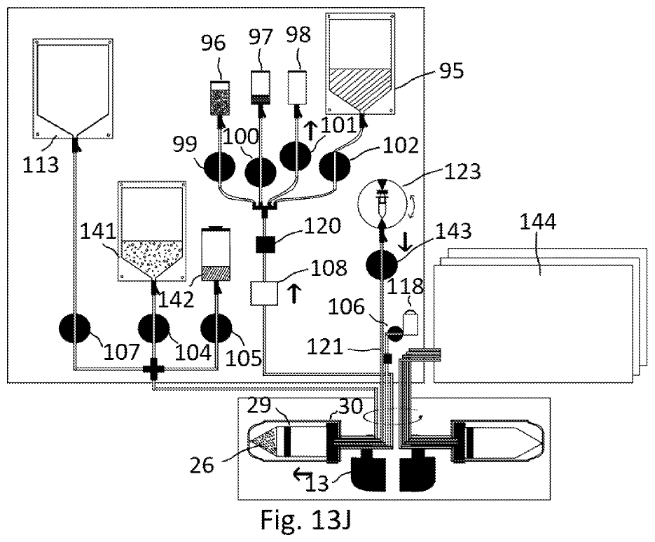

FIG. 13 shows the processes steps of the embodiment shown in FIG. 12.

Step 1 “Priming” (FIG. 13A): The pinch valve 106 opens and the hydraulic pump 123 primes the hydraulic tubing line 36 towards the degassing chamber 118. Once the tubing line 36 is properly primed, pinch valve 106 closes, pinch valve 143 opens, and hydraulic pump 123 actuates to move the piston 29 downwards within the processing chamber 26, such that piston 29 moves from the upper 27 to the lower body 28 of the processing chamber 26. As the piston 29 moves, the air trapped within the processing chamber 26 is pushed out towards the waste collection bag 95 when valve 102 is open.

Step 2 “Blood in” (FIG. 13B): Vertical position of the cassette 16 is employed while loading the blood sample, density gradient separation media 52, microbubbles 55 or any other solution/reagent into the processing chamber 26. Once the piston 29 is at the bottom of the processing chamber 26, pinch valve 104 and pinch valve 143 open to allow the hydraulic pump 123 to actuate the piston 29 to move upwards, thus letting blood flow from blood bag 141 into the chamber 26. An optical sensor 120 and a bubble air trapping detection and pressure monitoring sensor 121 may be used to detect colors and light absorbance to monitor erythrocytes 51 and other cells in the input tubing 38 or output tubing 40. This enables more accurate fractionation by PLAX system, and allows for the valve 104 and valve 143 to be opened and closed accordingly.

Step 3 “Density media in” (FIG. 13C): After the blood sample is loaded into the processing chamber 26, the density gradient separation media 52 located in density gradient separation media bag 142 is loaded into the chamber 26. At this point, the piston 29 may be disposed at the middle position of the chamber 26, although position may vary based on the selected processing protocol. By activating the hydraulic pump 123 to move the piston 29 upwards, and opening valve 105 and valve 143, the density gradient separation media 52 is carefully layered below the blood.

Step 4 “Centrifugation” (FIG. 13D): Centrifugation starts, and speed is stabilized according to the selected protocol (e.g., 2400 rpm), with all pinch valves, inlets, and outlets closed. A counter pressure may get established through the hydraulic pump 123 to keep piston 29 static within the processing chamber 26. The cassette 16 with the processing chamber 26 can swivel between a vertical position and a horizontal position due to centrifugation forces.

Step 5 “Erythrocyte collection” (FIG. 13E): After sedimentation time, the centrifuge 10 speed slowly decreases while maintaining the piston 29 at the same position within the processing chamber 26. Once the centrifuge 10 drops to a predefined speed, the piston 29 moves downwards at a pre-set speed (the value of which can be modified through the protocol/program parameters) via the hydraulic pump 123, for erythrocytes 51 collection and extraction, while maintaining valve 99 and valve 143 open, which respectively correspond to fraction collection container 96 and hydraulic pump 123. Erythrocyte 51 collection is optional, and alternatively erythrocytes 51 may be re-routed and extracted towards the waste bag 95.

Step 6 (FIG. 13F): A washing protocol is normally carried out between the different fraction collection steps by using buffer at buffer bag 113 to clean tubing running towards waste bag 95, therefore ensuring the highest purity across all the steps. The washing protocol is performed by actuating the pre-collection pump 108 and opening valves 102, 107 while maintaining the hydraulic pump 123 and pinch valve 143 closed. Upon completion of the washing protocol, fraction #2 (density gradient media 52), is extracted from the processing chamber 26 towards the waste bag 95 under centrifugation, by moving the piston 29 downwards with the hydraulic pump 123 and opening valve 102 and valve 143 which respectively correspond to waste bag 95 and hydraulic pump 123.

Step 7 (FIG. 13G): The speed of centrifugation decreases until reaching a stop, and the orientation of the processing chamber 26 is switched either within the cassette 16, or by flipping the cassette 16 within the rotor 13 either manually or automatedly. Centrifugation starts, and speed is stabilized according to the selected protocol. Due to the possibility of centrifugation in two different directions, fraction #4 (plasma 54) can be extracted from the processing chamber 26 under centrifugation, while retaining mononuclear cells 53. Plasma 54 transitions towards fraction bag 98 by moving the piston 29 downwards with the use of the hydraulic pump 123 and opening valve 101 and valve 143. Plasma 54 may be collected, or in the alternative, re-routed towards waste bag 95 and extracted.

When all steps described above are finalized, the speed of centrifugation slowly decreases until reaching a stop. Subsequently, all pinch valves close and pressure within the system stabilizes. Fraction containers 96, 97, 98 can be disconnected and the rest of the tubing set may be discarded if no further process is requested.

Step 8 (FIG. 13H): Mononuclear cells 53 retained within the processing chamber 26 may be used for subsequent processing, such as targeted microbubble-based cell isolation 56, T cell activation by APCs, transduction, and short-term cell culture, without leaving the processing chamber 26. (U.S. Pat. No. 10,479,976B2, US20220154150A1). For this selection process, with the cassette 16 at its resting vertical position, the centrifuge 10 stopped, and the processing chamber 26 in its original orientation, target microbubbles 55 may be introduced within the processing chamber 26 for binding. After interaction, a centrifugation protocol may be used to separate bound isolated cells (e.g., microbubbles 55 attached to target cells 146) from non-target cells 145 in the processing chamber 26. The non-targeted cells 146 can be expelled from the processing chamber 26 by moving the piston 29 downwards with the use of the hydraulic pump 123 either during centrifugation or by having the rotor 13 stationary. For this process, valve 102 and valve 143 open to expel the non-targeted cells 145 towards waste bag 95. A washing process may be performed to isolated target cells 53 by loading buffer from buffer bag 113 within the processing chamber 26 due to the movement of piston 29 upwards with the use of the hydraulic pump 123. After loading, mixing of the contents of the processing chamber 26 may be performed with the use of mixing device 24. This process may be performed repeatedly based on protocol.

Step 9 (FIG. 13.I): Alternatively, PLAX may be used solely for fraction separation and collection, therefore fraction #3 (mononuclear cells 53) can be extracted from the processing chamber 26 under centrifugation. The cells transition towards the fraction collection container 97 by moving the piston 29 downwards with the use of the hydraulic pump 123 and opening valve 100 and valve 143, which respectively correspond to fraction collection container 97 and hydraulic pump 123.

Step 10 (FIG. 13.J): Fraction #4 (plasma 54) can be extracted from the processing chamber 26 under centrifugation. The cells transition towards fraction container 98 by moving the piston 29 downwards with the use of the hydraulic pump 123 and opening valve 101 and valve 143, which respectively correspond to fraction container 98 and hydraulic pump 123. Plasma 54 may be collected, or in the alternative may be re-routed towards waste bag 95 and extracted.

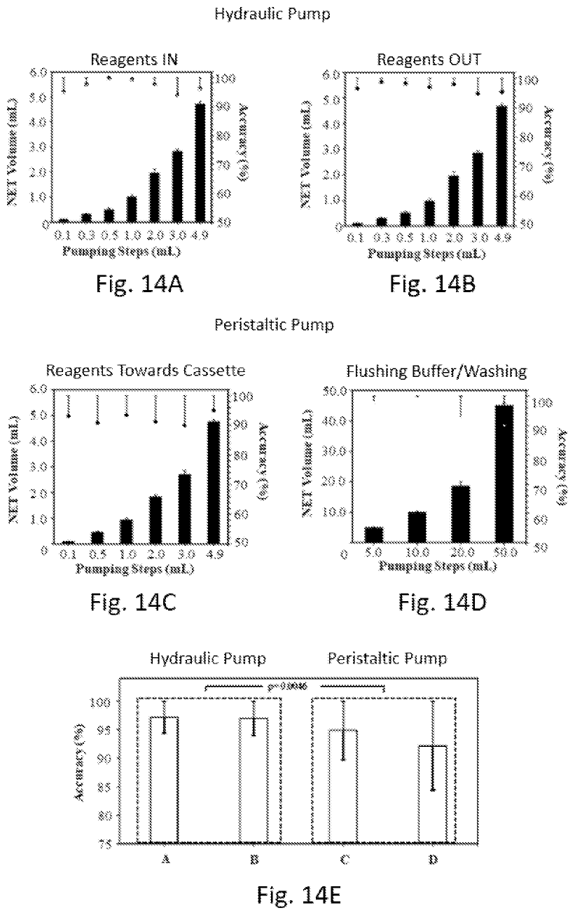

FIGS. 14A and 14B show bar plots graphing accuracy (%) over pumping steps (mL) for reagents in and reagents out, respectively, for a hydraulic pump 123. The reagents in accuracy for the hydraulic pump 123 at 4.9 mL was 97.22+2.78%, while reagents out accuracy for the hydraulic pump 123 was 96.88+3.02%.

FIGS. 14C and 14D show bar plots graphing accuracy (%) over pumping steps (mL) for reagents towards cassette 16 and flush PBS/washing, respectively, for a peristaltic pump 108. The reagents towards cassette 16 for the peristaltic pump 108 was 94.89+5.11%, while the flush PBS/washing accuracy was 92.20+7.80%.

FIG. 14E shows a comparison of the accuracy of the respective plots in FIGS. 14A-14D. The difference between groups is very significant, with a p-value <0.005. The volume control by hydraulic pump 123 is highly accurate (97.10+2.90%).

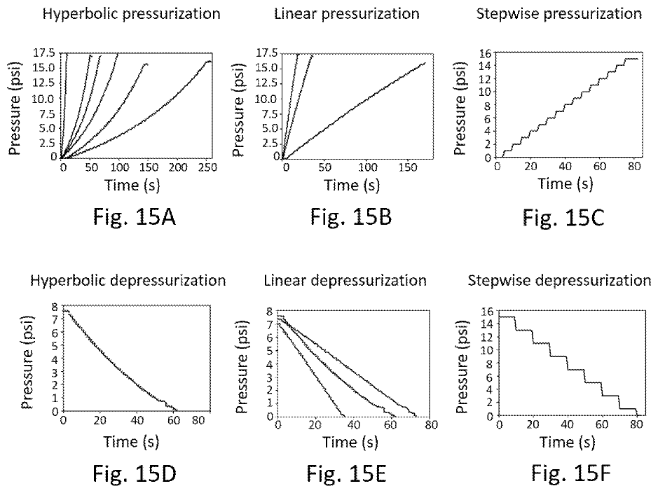

FIG. 15 displays exemplary plots of the different pressurization and depressurization profiles/patterns used when disrupting/dissolving microbubbles 55 bound to target cells 146. Depending on user needs or protocols, the processing chamber 26 may need to undergo a pre-mixing and/or constant mixing process for solution homogenization. Pressurization with a fixed or variable mixing speed may be achieved by the mixing device 24. The mixing speed may be fully controllable by the PLAX system, and may include up to 360-degree rotation. The speed of the piston 29 within the processing chamber 26 can be controlled and adjusted to provide full flexibility for microbubble target cell dissolution, and optimized automated parameters may be incorporated within the PLAX system.

The top left graph shows a plurality of hyperbolic pressurization curves for pressure (psi) over time (seconds). The respective curves show achieving a pressure from 0 psi to 15-17.5 psi for time intervals of 10, 50, 75, 100, 150, and 250 seconds.

The bottom left shows a hyperbolic depressurization plot for pressure (psi) over time (seconds), wherein pressure decreases from ˜7.5 psi to 0 psi within 60 seconds.

The top central graph shows a plurality of linear pressurization plots for pressure (psi) over time (seconds). The respective curves show achieving a pressure from 0 psi to 15-17.5 psi for time intervals of 25, 40, and 175 seconds.

The bottom central graph shows a plurality of linear depressurization plots for pressure (psi) over time (seconds). The respective curves show pressure decreases from 7-7.5 psi to 0 psi for time intervals of 35, 60, and 70 seconds.

The top right graph shows a stepwise pressurization plot for pressure (psi) over time (seconds) from 0 psi to 15 psi over 80 seconds.

The bottom right shows a stepwise depressurization plot for pressure (psi) over time (seconds) from 15 psi to 0 psi over 80 seconds.

The pressurization and depressurization profiles for this process are not limited to the use of the piston 29, and may fully adjustable when employing a compressor or pump during the process. These profiles may range from linear, stepwise, hyperbolic or a variation of these over time until the system reaches the required pressure (e.g., 1.5, 2, 2.5 atm) for partially or completely disrupting microbubbles 55. Following dissolution of microbubbles 55, targeted cells 146 return to the solution, which can be used for sequential steps. The sequential steps may include binding to another type of targeted microbubble, activation by aAPCs, or viral transduction mediated by aVPCs. The three steps can occur within the same processing chamber 26.

It should be noted that the PLAX invention can take on different forms or variations without straying from the fundamental essence of its key attributes. Therefore, all embodiments that fall within the scope and equivalence of the claims are intended to be encompassed by those claims.

The above description of the disclosed embodiments and applications is provided to enable any person skilled in the art to make or use the present disclosure. Various modifications to these embodiments will be readily apparent to those skilled in the art, and the generic principles described herein can be applied to other embodiments without departing from the spirit or scope of the present disclosure. Thus, it is to be understood that the description and drawings presented herein represent a presently preferred embodiment of the present disclosure and are therefore representative of the subject matter, which is broadly contemplated by the present disclosure. It is further understood that the scope of the present disclosure fully encompasses other embodiments that may become obvious to those skilled in the art.

The following is a list of reference numerals as shown in the drawings:
1 tubing
2 spring adapter
3 tube locking input
4 arm housing
ring enclosed structure input
6 ring enclosed structure output
7 stoppers
9 counterweight structure
centrifuge
11 Central centrifuge housing
12 rotational element
13 rotor
14 single motor
arms
16 swinging bucket cassette
17 locking mechanism
18 tube diverter
19 tube locking output
retaining channels
21 frame
22 ultrasonic sensors
23 built-in triple axis accelerometer
24 mixing device
25 rotor bucket supports
26 processing chamber
27 upper body (processing chamber)
28 lower body (processing chamber)
29 piston
30 cap
31 O-ring
32 tubing connector (upper body)
33 tubing connector (lower body)
34 inlet cavities
35 outlet cavities
36 hydraulic tubing
38 input tubing
39 input retaining channels
output tubing
41 output retaining channels
42 hinge
43 pattern
44 reciprocal cassette counterpart
reciprocal cassette
46 pocket
47 bushing
48 physical support
49 counterpart pattern
release latch mechanism
51 erythrocytes (RBCs)
52 density gradient media
53 mononuclear cells
54 plasma
55 microbubbles
56 targeted microbubble-based cell isolation
57 moving/extending/reaching structure
58 cassette bottom
59 movable pressurization platform
60 linear rail shaft optical axis rod
61 lead screw
62 shaft coupling connector
63 stepper motor
64 top support structure
65 bottom support structure
66 rotational elements
67 sensor cavity-coupling integrations
68 weight cells
69 syringe attachment
70 flange
71 holder
72 barrel flange
73 processing buffer container
74 plunger
75 stepper motor support structure
76 rotational elements
77 lead screw translation adapter
78 plurality of holes
79 plurality of holes
80 indentations
81 rod mixer
82 clamp mechanism
83 multiple input reagent containers
84 ceiling cap
85 filter
86 tubing connector
87 rotational elements
88 support structure
89 connector/adapter
90 stepper motor
91 stepper motor housing
92 housing
93 sensor
94 hook connector
95 waste bag
96 fraction collection container
97 fraction collection container
98 fraction collection container
99 individual external pinch valve
100 multichannel external pinch valve
101 multichannel external pinch valve
102 valve
103 pre-collection pinch valve
104 pinch valve
105 valve
106 degassing pinch valve
107 buffer pinch valve
108 pre-collection peristaltic pump
109 housing adapter
110 retaining channels
111 housing adapter
112 housing adapter
113 buffer bag
114 hook connector
115 sensor
116 housing
117 intuitive user interface screen
118 degassing chamber
119 filter
120 optical sensor
121 Bubble air trapping detection and pressure monitoring sensor
122 tube welding subsystem
123 hydraulic pump
124 pre-mixing subsystem
125 dynamic focusing platform
126 microfluidic chamber
127 microfluidic chamber inlet
128 microfluidic chamber outlet
129 stepper motor
130 shaft coupling connector
131 lead screw
132 linear motion axis rail
133 monitoring subsystem top support structure
134 monitoring subsystem bottom support structure
135 rail clamps
136 light source
137 imaging device
138 monitoring subsystem middle support structure
139 microfluidic chamber connector
140 microfluidic chamber connector
141 blood bag
142 density gradient separation media bag
143 hydraulic pinch valve
144 control/electronic module
145 non-target cells
146 targeted cells

What is claimed is:

1. A closed system for blood sample processing to separate biological components, said system comprising:
a centrifuge having a rotor, said centrifuge being configured to centrifugate a blood sample;
a processing chamber attached to the rotor, said processing chamber having a first end and a second end, and is configured to contain at least one blood sample;
a piston housed in the processing chamber, said piston configured to move between the first position closer to a first end of the processing chamber and second position closer to a second end of the processing chamber, the second end being located opposite of the first end; and
a plurality of tubes connecting the processing chamber to the at least one blood sample;
wherein actuation of the piston making the piston move dispels fractions of the blood sample during centrifugation or at rest to at least one fraction bag.

2. The system according to claim 1, wherein at least two swinging bucket cassettes are hinged by respective hinges on opposite sides of rotor, said swinging bucket cassettes accommodating the processing chamber, and the range of motion is at least between a horizontal orientation of the swinging bucket cassettes and with it of the processing chamber, which horizontal position is assumed during centrifugation, and a vertical position, wherein said vertical position is assumed while no centrifugation is performed.

3. The system according to claim 1, wherein the processing chamber has a first end and a second end that is opposite to the first end, and the swinging bucket cassette has a first end and a second end and is configured to receive the processing chamber is a first orientation with a first end of the processing chamber being located at the first end of the swinging bucket cassette; and is configured to receive the processing chamber in a second orientation with the second end of the processing chamber being located at the first end of the swinging bucket cassette.

4. The system according to claim 1, wherein the processing chamber is removably held in the swinging bucket cassette, which comprises a first swinging bucket cassette part that can be separated from a second swinging bucket cassette part to open the cassette for inserting or removing the processing chamber, and further comprises a releasable locking mechanism configured to interface with the swinging bucket cassette for holding the first and second swinging bucket cassette parts together.

5. The system according to claim 1, further comprising a peristaltic pump configured to route, re-direct, and/or move liquid from the processing chamber to a waste collection bag.

6. The system according to claim 1, wherein the processing chamber is cylindrical and the piston is a free-piston dividing the processing chamber into a first processing chamber part facing the blood sample and a second processing chamber part to which hydraulic fluid is fed or withdrawn by a hydraulic pump for moving the piston between said first and second positions.

7. The system according to claim 1, wherein the hydraulic pump employs a stepper motor coupled to a leadscrew via a shaft coupling connector to drive a plunger-flange within a processing buffer container to feed hydraulic fluid through a hydraulic tubing into the second processing chamber part.

8. The system according to claim 1, further comprising a pre-mixing subsystem comprising:
at least one input reagent container having a first end and a second end, wherein the first end is configured to interface with tubing, and the second end is configured to be sealed via a sealing cap;
at least one tube configured to connect with the first end of the at least one input container; and
a rod mixer driven by a stepper motor, said rod mixer configured to mix the contents of the at least one input reagent container; wherein
the least one tube connects the pre-mixing subsystem to the processing chamber.

9. The system according to claim 1, further comprising in connection with the processing chamber via tubing a blood container, a density gradient separation media bag in connection with the processing chamber via tubing, a buffer bag, and at least one collection/waste bag.

10. A method of cell selection from a blood sample, comprising:
a) priming the system by removing air from processing chamber through actuation of piston in a first direction via a hydraulic pump;
b) pumping blood into the processing chamber, wherein said processing chamber is housed in a swinging bucket cassette;
c) pumping density gradient separation media into the processing chamber;
d) actuating centrifugal rotation of rotor to which the swinging bucket cassette is attached; and
while performing centrifugation, moving a piston within the processing chamber, performing in sequence the following steps:
e) extracting erythrocytes from the processing chamber;
f) extracting density gradient separation media from the processing chamber;
g) extracting mononuclear cells from the processing chamber;
h) extracting plasma from the processing chamber;
i) ceasing centrifugation, closing valves, and stabilizing pressure.

11. A method of cell selection from a blood sample, comprising:
a) priming the system by removing air from processing chamber through actuation of piston in a first direction via a hydraulic pump;
b) pumping blood into the processing chamber, wherein said processing chamber is housed in a swinging bucket cassette;
c) pumping density gradient separation media into the processing chamber;
d) actuating centrifugal rotation of rotor to which the swinging bucket cassette is attached; and
while performing centrifugation, with the processing chamber in a horizontal orientation, moving a piston within the processing chamber, performing in sequence the following steps:
e) extracting erythrocytes from the processing chamber;
f) extracting density gradient separation media from the processing chamber;
g) stopping centrifugation;
h) flipping an orientation of the processing chamber with respect to the rotor so that an end of the processing chamber that was proximal to the rotor now becomes the free distal end of the processing chamber and an end of the processing chamber that was the distal free end of the processing chamber now become proximal to the rotor;
i) extracting plasma from the processing chamber;
j) ceasing centrifugation, closing valves, and stabilizing pressure, leaving mononuclear cells in the processing chamber.

12. The method of claim 11, further including the step of:
k) bringing the processing chamber into a vertical position
l) binding the mononuclear cells in the processing chamber with microbubbles attaching to a specific marker of a first subset of the mononuclear cells to be selected;
m) discarding the non-selected mononuclear cells by extracting these from the processing chamber;
n) disrupting the microbubbles by increasing the pressure in the processing chamber;
o) binding the first subset of the mononuclear cells in the processing chamber with microbubbles attaching to a specific marker of a second subset of the mononuclear cells to be selected out of the first subset of the mononuclear cells; and
p) discarding the non-selected mononuclear cells by extracting these from the processing chamber.

13. The method of claim 10, further including the step of: monitoring by using at least one of an optical sensor, a bubble air trapping detection and pressure monitoring sensor to detect colors and light absorbance to monitor erythrocytes and other cells.

14. The method of claim 11, further including the step of: monitoring by using at least one of an optical sensor, a bubble air trapping detection and pressure monitoring sensor to detect colors and light absorbance to monitor erythrocytes and other cells.

15. The method according to claim 11, further comprising the step of:
performing at least one of the following subsequent processes on the mononuclear cells in the processing chamber: targeted microbubble-based cell isolation, T cell activation, transduction, and short-term cell culture.
